(19) United States
(12) Patent Application Publication
(10) Pub. No.: US 2025/0195417 A1
(43) Pub. Date: Jun. 19, 2025

(54) ADDITIVE COMPOSITION FOR ORALLY DISINTEGRATING TABLET

(71) Applicant: Daicel Corporation, Osaka-shi, Osaka (JP)

(72) Inventors: Tomohito OKABAYASHI, Tokyo (JP); Yoshihisa TAKIGAWA, Tokyo (JP); Naohiro HASHIKAWA, Tokyo (JP)

(73) Assignee: Daicel Corporation, Osaka-shi, Osaka (JP)

(21) Appl. No.: 18/846,235

(22) PCT Filed: Mar. 22, 2023
(86) PCT No.: PCT/JP2023/011140

§ 371 Date: Sep. 11, 2024

(30) Foreign Application Priority Data
Mar. 22, 2022 (JP) 2022-046074

(51) Int. Cl. A61K 9/00; A61K 9/20

(52) U.S. Cl. CPC A61K 9/0056; A61K 9/2018; A61K 9/2027; A61K 9/2054

(57) ABSTRACT

The present disclosure provides an additive formulation for an orally disintegrating tablet containing (A) an excipient, (B) a crystalline cellulose, (C) a swellable water-insoluble polymer, and (D) (D1) a (meth)acrylic polymer and/or (D2) a cellulosic polymer.

### TECHNICAL FIELD

The present disclosure relates to an additive composition for an orally disintegrating tablet, an orally disintegrating tablet composition containing the additive composition, and an orally disintegrating tablet.

### BACKGROUND ART

Orally disintegrating tablets can be safely taken by patients, elderly people, children, and others who have difficulty swallowing drugs, and because orally disintegrating tablets are in a highly convenient form in which they can be taken without water, such tablets are also useful for young and middle-aged people who work. Similar to ordinary tablets, orally disintegrating tablets require, as basic characteristics, a sufficient breaking strength (tablet hardness) to prevent tablet chipping, pulverization, or the like during tablet production, during transport, or during unsealing of the medication, and also require a short disintegration time (disintegrability) to achieve rapid tablet disintegration in the oral cavity.

Meanwhile, high tablet hardness and short disintegration time are mutually contradictory properties, and in general, when the molding pressure is increased to enhance the hardness, the disintegration time tends to be lengthened because voids inside the tablet are reduced, and when the molding pressure is decreased to shorten the disintegration time, the hardness tends to be reduced due to insufficient bonding between particles. For this reason, various techniques have been developed to achieve compatibility or optimum balance between these two properties.

For example, Patent Document 1 discloses a method for producing a disintegrating particle composition containing three components; i.e., a first disintegrant component formed from an acidic carboxymethyl cellulose, a second disintegrant component other than the acidic carboxymethyl cellulose, and an excipient, the production method including a first wet granulation process involving the use of any two components among the three components, and a second wet granulation process involving the use of at least a granulated product obtained in the first wet granulation process and the remaining one component not used in the first wet granulation process. Patent Document 1 also describes that a disintegrating particle composition produced by this production method can increase tablet hardness while maintaining a short disintegration time, even when a crystalline cellulose is added.

Patent Document 2 describes that a disintegrating particle composition containing four components; i.e., a first disintegrant component containing an acidic carboxymethyl cellulose, a second disintegrant component other than the acidic carboxymethyl cellulose (specifically, one or more components selected from crospovidone, croscarmellose sodium, sodium carboxymethyl starch, low-substituted hydroxypropyl cellulose, calcium carboxymethyl cellulose, hydroxypropyl starch and starch), an excipient formed of a sugar or a sugar alcohol, and a crystalline cellulose exhibits a short disintegration time while having a tablet hardness comparable to that of a disintegrating particle composition containing a crystalline cellulose.

### CITATION LIST

### Patent Document

Patent Document 1: WO 2013/146917
Patent Document 2: WO 2014/046035

### SUMMARY OF INVENTION

### Technical Problem

The high level of convenience of an orally disintegrating tablet is extremely useful for all age groups including the elderly, and therefore the demand for orally disintegrating tablets is expected to increase. Meanwhile, orally disintegrating tablets are required to have mutually contradicting properties including a tablet hardness that is preferably improved and a disintegration time that is preferably shortened. Therefore, orally disintegrating tablets are desirably provided with properties whereby a higher tablet hardness than ever before is obtained at the same tableting pressure, the same tablet hardness is obtained at a lower tableting pressure than ever before, or a higher tablet hardness is obtained at a lower tableting pressure, or in other words, orally disintegrating tablets are desirably provided with higher moldability.

Thus, an object of the present disclosure is to provide an additive formulation for an orally disintegrating tablet that can achieve higher moldability.

### Solution to Problem

As a result of intensive studies, the present inventors have found that moldability can be improved by blending a (meth)acrylic polymer and/or a predetermined cellulosic polymer into an additive composition for an orally disintegrating tablet, the additive composition containing a predetermined excipient, a crystalline cellulose, and a predetermined swellable water-insoluble polymer. The present disclosure has been achieved through further examinations based on this finding.

Specifically, the present disclosure provides the following aspects.
Aspect 1. An additive composition for an orally disintegrating tablet, the additive composition containing:
(A) an excipient selected from the group consisting of a water-soluble saccharide and a sugar alcohol;
(B) a crystalline cellulose;
(C) a swellable water-insoluble polymer selected from the group consisting of crospovidone and croscarmellose sodium; and
(D) (D1) a (meth)acrylic polymer and/or (D2) a cellulosic polymer selected from the group consisting of ethyl cellulose, cellulose acetate, hydroxypropylmethyl cellulose acetate succinate, and hydroxypropylmethyl cellulose phthalate.
Aspect 2. The additive composition for an orally disintegrating tablet according to aspect 1, wherein the component (D1) is a (meth)acrylic copolymer containing [d11] an alkyl (meth)acrylate unit and [d12] a comonomer unit selected from the group consisting of (meth)acrylic acid, an alkyl (meth)acrylate, an aminoalkyl (meth)acrylate, and an ammonioalkyl (meth)acrylate.
Aspect 3. The additive composition for an orally disintegrating tablet according to aspect 1 or 2, wherein a content of the component (D) per 1 part by weight of a total amount of the component (A) and the component (B) is from 0.01 to 5 parts by weight.
Aspect 4. The additive composition for an orally disintegrating tablet according to any one of aspects 1 to 3, wherein a content of the component (D) per 1 part by weight of the component (C) is from 0.1 to 50 parts by weight.
Aspect 5. The additive composition for an orally disintegrating tablet according to any one of aspects 1 to 4, wherein a content of the component (D) is from 1 to 70 wt. %.
Aspect 6. An orally disintegrating tablet composition containing the additive composition for an orally disintegrating tablet described in any one of aspects 1 to 5 and (E) an active pharmaceutical ingredient.
Aspect 7. The orally disintegrating tablet composition according to aspect 6, wherein a content of the component (E) per 100 parts by weight of the additive composition is from 0.5 to 233 parts by weight.
Aspect 8. An orally disintegrating tablet containing the orally disintegrating tablet composition described in aspect 6 or 7.
Aspect 9. A method for producing an orally disintegrating tablet, the method including:
preparing an orally disintegrating tablet composition by mixing (A) an excipient selected from the group consisting of a water-soluble saccharide and a sugar alcohol, (B) a crystalline cellulose, (C) a swellable water-insoluble polymer selected from the group consisting of crospovidone and croscarmellose sodium, (D) (D1) a (meth)acrylic polymer and/or (D2) a cellulosic polymer selected from the group consisting of ethyl cellulose, cellulose acetate, hydroxypropylmethyl cellulose acetate succinate, and hydroxypropylmethyl cellulose phthalate, and (E) an active pharmaceutical ingredient; and
tableting the orally disintegrating tablet composition.
Aspect 10. The method according to aspect 9, wherein the tableting is carried out at a tableting pressure of from 20 to 600 MPa.

### Advantageous Effects of Invention

According to the present disclosure, there is provided an additive formulation for an orally disintegrating tablet that can achieve higher moldability.

### DESCRIPTION OF EMBODIMENTS

### 1. Additive Composition for Orally Disintegrating Tablet

The additive composition for an orally disintegrating tablet according to an embodiment of the present disclosure is characterized by containing (A) an excipient (hereinafter, also referred to as the “component (A)”) selected from the group consisting of a water-soluble saccharide and a sugar alcohol; (B) a crystalline cellulose (hereinafter, also referred to as the “component (B)”); (C) a swellable water-insoluble polymer (hereinafter, also referred to as the “component (C)” or the “predetermined swellable water-insoluble polymer”) selected from the group consisting of crospovidone and croscarmellose sodium; and (D) (D1) a (meth)acrylic polymer (hereinafter, also referred to as the “component (D1)”) and/or (D2) a cellulosic polymer (hereinafter, also referred to as the “component (D2)” or the “predetermined cellulosic polymer”) selected from the group consisting of ethyl cellulose, cellulose acetate, hydroxypropylmethyl cellulose acetate succinate, and hydroxypropylmethyl cellulose phthalate (these components (D1) and (D2) may be collectively referred to as the “component (D)”). Hereinafter, embodiments of the additive composition for an orally disintegrating tablet of the present disclosure will be described in detail.

### 1-1. (A) Excipient

The additive composition for an orally disintegrating tablet according to an embodiment of the present disclosure contains, as the component (A), an excipient selected from the group consisting of a water-soluble saccharide and a sugar alcohol.

Examples of the water-soluble saccharide include disaccharides such as trehalose, lactose, and maltose. Examples of the sugar alcohol include mannitol, erythritol, sorbitol, maltitol, and xylitol. A single type of these excipients may be used alone, or a plurality of types may be used in combination. Among these excipients, from the viewpoint of further improving the moldability of the orally disintegrating tablet, a water-soluble saccharide is preferred, a disaccharide is more preferred, and mannitol is even more preferred.

The content of the component (A) contained in the additive composition for an orally disintegrating tablet according to an embodiment of the present disclosure is, for example, from 5 to 99 wt. %, preferably from 10 to 95 wt. %, more preferably from 15 to 90 wt. %, even more preferably from 15 to 85 wt. %, and yet even more preferably from 15 to 80 wt. %, from 15 to 75 wt. %, from 15 to 70 wt. %, from 15 to 65 wt. %, or from 15 to 60 wt. %. Furthermore, the lower limit of the range of the content of the component (A) may be 20 wt. % or more, 25 wt. % or more, 30 wt. % or more, 35 wt. % or more, or 40 wt. % or more, and the upper limit of the range of the content of the component (A) may be 55 wt. % or less, or 50 wt. % or less.

### 1-2. (B) Crystalline Cellulose

The additive composition for an orally disintegrating tablet according to an embodiment of the present disclosure contains a crystalline cellulose as the component (B). The crystalline cellulose to be used may be a crystalline cellulose known to a person skilled in the art. Examples of the crystalline cellulose that can be used in the present disclosure include commercially available products such as Avicel (FMC Corporation), Ceolus (Asahi Kasei Corporation), and Vivapur (J. Rettenmaier & Sohne GmbH).

The crystalline cellulose used as the component (B) may be prepared by further micronizing a commercially available crystalline cellulose. The micronizing method is not particularly limited, and a typically known micronizing method can be used. Examples of the method for micronizing crystalline cellulose include a method of directly pulverizing crystalline cellulose fibers in a dry state using a ball mill to prepare micronized crystalline cellulose, and a method including a process of microfibrillating an aqueous dispersion of crystalline cellulose fibers using a high-pressure homogenizer to prepare an aqueous suspension, a process of subjecting the aqueous suspension to solvent replacement, a process of removing the solvent, and a process of pulverizing the residue to prepare micronized crystalline cellulose.

The content of the component (B) contained in the additive composition for an orally disintegrating tablet according to an embodiment of the present disclosure is, for example, from 0.5 to 60 wt. %, preferably from 1 to 50 wt. %, more preferably from 5 to 45 wt. %, even more preferably from 7 to 40 wt. %, and still more preferably from 10 to 37 wt. %. Furthermore, the lower limit of the range of the content of the component (B) may be 15 wt. % or more, 20 wt. % or more, 25 wt. % or more, or 35 wt. % or more, and the upper limit of the range of the content of component (B) may be 35 wt. % or less, 30 wt. % or less, 25 wt. % or less, or 20 wt. % or less.

### 1-3. (C) Predetermined Swellable Water-Insoluble Polymer

The additive composition for an orally disintegrating tablet according to an embodiment of the present disclosure contains, as the component (C), a swellable water-insoluble polymer selected from the group consisting of crospovidone and croscarmellose sodium.

Crospovidone is a crosslinked polymer of 1-vinyl-2-pyrrolidone, and croscarmellose sodium is a crosslinked product of sodium carboxymethyl cellulose.

The water swelling rate of the component (C) is, for example, more than 60%, preferably 70% or more, and more preferably 78% or more, 100% or more, 200% or more, 300% or more, 400% or more, 500% or more, 600% or more, or 700% or more. The upper limit of the water swelling rate is not particularly limited, and may be, for example, 800% or less, 700% or less, 600% or less, 500% or less, 400% or less, 300% or less, 200% or less, or 100% or less.

The water swelling rate is a value measured by the following method.

### Method for Calculating Water Swelling Rate

75 mL of purified water is added to a beaker, 5.0 g of a sample is then added with stirring, and the mixture is stirred for 3 minutes. The suspension is transferred to a 100 mL measuring cylinder, and the volume is increased to 100 mL, after which the suspension is left to stand for 16 hours, and then the volume after swelling is read. The read volume is substituted into the following formula to calculate the swelling rate.

$$Swelling rate [ \% ] = ( volume [ mL ] after swelling - volume [ mL ] before swelling ) / \\ ( volume [ mL ] before swelling ) \times 100 \qquad [ Math . 1 ]$$

A single type of these predetermined swellable water-insoluble polymers may be used alone, or a plurality of types may be used in combination.

The additive composition for an orally disintegrating tablet according to an embodiment of the present disclosure in which crospovidone is contained as the component (C) from among these predetermined swellable water-insoluble polymers is preferred in that the effect of improving moldability is particularly high as compared with a case in which the component (D) is not contained.

The content of the component (C) contained in the additive composition for an orally disintegrating tablet according to an embodiment of the present disclosure is, for example, from 0.1 to 20 wt. %, preferably from 0.5 to 15 wt. %, more preferably from 1 to 10 wt. %, and even more preferably from 2 to 9 wt. %.

The content of the component (C) contained in the additive composition for an orally disintegrating tablet according to an embodiment of the present disclosure is, for example, from 3 to 16 parts by weight, preferably from 5 to 14 parts by weight, more preferably from 7 to 12 parts by weight, and still more preferably from 9 to 10 parts by weight per 100 parts by weight of the total amount of the components (A), (B), and (C).

### 1-4. (D) (Meth)Acrylic Polymer or Predetermined Cellulosic Polymer

The additive composition for an orally disintegrating tablet according to an embodiment of the present disclosure contains, as the component (D), (D1) a (meth)acrylic polymer and/or (D2) a cellulosic polymer selected from the group consisting of ethyl cellulose, cellulose acetate, hydroxypropylmethyl cellulose acetate succinate, and hydroxypropylmethyl cellulose phthalate.

In the additive composition for an orally disintegrating tablet according to an embodiment of the present disclosure, the component (D) to be used may be either the component (D1) or the component (D2), or both of these in combination. Moreover, for each of the components (D1) and (D2), a single type from each of the components (D1) and (D2) described below may be used alone, or two or more types may be used in combination.

### 1-4-1. (D1) (Meth)Acrylic Polymer

“(Meth)acrylic” is a term encompassing both “methacrylic” and “acrylic”. The (meth)acrylic polymer serving as the component (D1) may be either a copolymer or a homopolymer as long as the (meth)acrylic polymer contains a (meth)acrylic acid monomer unit and/or a (meth)acrylate monomer unit.

The copolymer of the (meth)acrylic polymer is not particularly limited, and examples include (meth)acrylic copolymers containing [d1] an alkyl (meth)acrylate monomer unit (hereinafter, also referred to as the “unit [d1]”) and [d2] a comonomer unit (hereinafter, also referred to as the “unit [d2]”) selected from the group consisting of (meth)acrylic acid, an alkyl (meth)acrylate, an aminoalkyl (meth)acrylate, and an ammonioalkyl (meth)acrylate.

That is, specific examples of the (meth)acrylic copolymer include the following:
(D11) a (meth)acrylic copolymer (hereinafter, also referred to as the “component (D11)”) containing an alkyl (meth)acrylate unit as the unit [d1], and (meth)acrylic acid as the unit [d2];
(D12) a (meth)acrylic copolymer (hereinafter, also referred to as the “component (D12)”) containing an alkyl (meth)acrylate unit as the unit [d1], and an alkyl (meth)acrylate as the unit [d2];
(D13) a (meth)acrylic copolymer (hereinafter, also referred to as the “component (1D3)”) containing an alkyl (meth)acrylate unit as the unit [d1], and an aminoalkyl (meth)acrylate as the unit [d2]; and
(D14) a (meth)acrylic copolymer (hereinafter, also referred to as the “component (D14)”) containing an alkyl (meth)acrylate unit as the unit [d1], and an ammonioalkyl (meth)acrylate as the unit [d2].

In the (meth)acrylic copolymer, each of the unit [d1] and the unit [d2] may contain a single unit alone or a combination of a plurality of units.

### 1-4-1-1. Component (D11)

In the component (D11), which is a (meth)acrylic copolymer containing an alkyl (meth)acrylate unit as the unit [d1] and (meth)acrylic acid as the unit [d2], the alkyl group constituting the alkyl ester contained in the unit [d1] may be a lower alkyl group, and examples thereof include alkyl groups having from 1 to 6 carbons, preferably from 1 to 5 carbons, more preferably from 1 to 4 carbons, even more preferably from 1 to 3 carbons, and still even more preferably 1 or 2 carbons.

Preferred examples of the component (D11) include a copolymer of a lower alkyl methacrylate and methacrylic acid, a copolymer of methyl methacrylate and methacrylic acid, and a copolymer of ethyl acrylate and methacrylic acid.

The polymerization ratio between the unit [d1] and the unit [d2] in the component (D11) is not particularly limited. For example, the amount of the unit [d2] relative to 1 mol of the unit [d1] is from 0.5 to 1.5 mol, preferably from 0.8 to 1.2 mol, and more preferably from 0.9 to 1.1 mol.

Examples of commercially available products of the component (D11) include Eudragit (registered trademark) type L, which is a copolymer of an alkyl methacrylate and methacrylic acid at a molar ratio of 1:1, Eudragit (registered trademark) type L, which is a copolymer of methyl methacrylate and methacrylic acid at a molar ratio of 1:1, Eudragit (registered trademark) type L-55, which is a copolymer of ethyl acrylate and methacrylic acid at a molar ratio of 1:1, Eudragit (registered trademark) type S, which is a copolymer of methyl methacrylate and methacrylic acid at a molar ratio of 1:0.4, and Eudragit (registered trademark) type FS, which is a copolymer of methyl methacrylate, methyl acrylate, and methacrylic acid at a polymerization ratio of 1:3:0.5.

### 1-4-1-2. Component (D12)

In the component (D12), which is a (meth)acrylic copolymer containing an alkyl (meth)acrylate unit as the unit [d1] and an alkyl (meth)acrylate as the unit [d2], the unit [d1] and the unit [d2] are units having mutually different structures.

The alkyl group constituting the alkyl ester contained in the unit [d1] of the component (D12) may be a lower alkyl group, and examples thereof include alkyl groups having from 1 to 6 carbons, preferably from 1 to 5 carbons, more preferably from 1 to 4 carbons, and even more preferably from 1 to 3 carbons.

The alkyl group constituting the alkyl ester contained in the unit [d2] of the component (D12) may be a lower alkyl group different from that of the unit [d1], and examples thereof include alkyl groups having from 1 to 6 carbons, preferably from 1 to 5 carbons, more preferably from 1 to 5 carbons, even more preferably from 1 to 3 carbons, and yet even more preferably 1 or 2 carbons.

Examples of commercially available products of the component (D12) include Eudragit (registered trademark) type NE type, which is a copolymer of ethyl acrylate and methyl methacrylate at a molar ratio of 1:2.3.

### 1-4-1-3. Component (D13)

In the component (D13), which is a (meth)acrylic copolymer containing an alkyl (meth)acrylate unit as the unit [d1] and an aminoalkyl (meth)acrylate as the unit [d2], the alkyl group constituting the alkyl ester contained in the unit [d1] may be a lower alkyl group, and examples thereof include alkyl groups having from 1 to 6 carbons, preferably from 1 to 5 carbons, and more preferably from 1 to 4 carbons.

The aminoalkyl group constituting the unit [d2] of the component (D13) may be a lower alkyl group substituted with an amino group, and examples thereof include aminoalkyl groups having from 1 to 6 carbons, preferably from 1 to 5 carbons, more preferably from 1 to 5 carbons, still more preferably from 1 to 3 carbons, yet even more preferably 1 or 2 carbons, and particularly preferably 2 carbons. The amino group may have a substituent. Examples of the substituent include lower alkyl groups, and more specific examples include alkyl groups having from 1 to 6 carbons, preferably from 1 to 5 carbons, more preferably from 1 to 5 carbons, even more preferably from 1 to 3 carbons, still more preferably 1 or 2 carbons, and most preferably 1 carbon. The number of substituents in the amino group is, for example, 1 or 2, and is preferably 2. That is, preferred examples of the amino group having a substituent include a monoalkylamino group and a dialkylamino group (preferably a dialkylamino group).

Preferred examples of the component (D13) include copolymers of butyl methacrylate and methyl methacrylate with (2-dimethylaminoethyl) methacrylate.

The polymerization ratio between the unit [d1] and the unit [d2] in the component (D13) is not particularly limited. For example, the amount of the unit [d2] relative to 1 mol of the unit [d1] is from 0.5 to 1.5 mol, preferably from 0.8 to 1.2 mol, and more preferably from 0.9 to 1.1 mol.

Examples of commercially available products of the component (D13) include Eudragit (registered trademark) EPO, which is a copolymer of butyl methacrylate, (2-dimethylaminoethyl) methacrylate, and methyl methacrylate at a molar ratio of 1:2:1.

### 1-4-1-4. Component (D14)

In the component (D14), which is a (meth)acrylic copolymer containing an alkyl (meth)acrylate unit as the unit [d1] and an ammonioalkyl (meth)acrylate as the unit [d2], the alkyl group constituting the alkyl ester contained in the unit [d1] may be a lower alkyl group, and examples thereof include alkyl groups having from 1 to 6 carbons, preferably from 1 to 5 carbons, more preferably from 1 to 5 carbons, and even more preferably from 1 to 3 carbons.

The ammonioalkyl (meth)acrylate serving as the unit [d2] of the component (D14) is a quaternary ammonium salt of the aminoalkyl (meth)acrylate serving as the unit [d2] of the component (D13). That is, in the ammonioalkyl (meth)acrylate, the ammonio group may have a substituent. Examples of the substituent include lower alkyl groups, and more specific examples include alkyl groups having from 1 to 6 carbons, preferably from 1 to 5 carbons, more preferably from 1 to 5 carbons, even more preferably from 1 to 3 carbons, still more preferably 1 or 2 carbons, and most preferably 1 carbon. The number of substituents in the ammonio group may be 1, 2 or 3, and is preferably 3. That is, preferred examples of the ammonio group having a substituent include a monoalkylammonio group, a dialkylammonio group, and a trialkylammonio group (preferably a trialkylammonio group). The counter anion of the ammonioalkyl (meth)acrylate is not particularly limited, and may be any anion that stabilizes the ammonio group, but a chloride ion is preferred.

Preferred examples of the component (D14) include copolymers of ethyl acrylate and methyl methacrylate with trimethyl ammonioethyl methacrylate chloride.

The polymerization ratio between the unit [d1] and the unit [d2] in the component (D14) is not particularly limited. For example, the amount of the unit [d2]relative to 1 mol of the unit [d1] is from 0.01 to 0.1 mol, preferably from 0.02 to 0.08 mol, and more preferably from 0.03 to 0.07 mol.

Commercially available products of the component (D14) include Eudragit (registered trademark) RL, which is a copolymer of ethyl acrylate, methyl methacrylate, and trimethyl ammonioethyl methacrylate chloride at a molar ratio of 1:2:0.2, Eudragit (registered trademark) RS, which is a copolymer of ethyl acrylate, methyl methacrylate, and trimethyl ammonioethyl methacrylate chloride at a molar ratio of 1:2:0.1, and Eudragit (registered trademark) RD, which is a copolymer of ethyl acrylate, methyl methacrylate, and trimethyl ammonioethyl methacrylate chloride at a molar ratio of 1:2:0.2 containing carboxymethyl cellulose in combination.

### 1-4-2. (D2) Predetermined Cellulosic Polymer

The predetermined cellulosic polymer serving as the component (D2) is selected from the group consisting of ethyl cellulose, cellulose acetate, hydroxypropylmethyl cellulose acetate succinate (HPMCAS), and hydroxypropylmethyl cellulose phthalate (HPMCP).

The ethoxyl group content of ethyl cellulose is, for example, from 44.0 to 51.0 wt. %.

The acetate group content of cellulose acetate is, for example, from 29.0 to 44.8 wt. %.

The hydroxypropylmethyl cellulose acetate succinate (HPMCAS) has a methoxyl group content of, for example, from 18 to 28 wt. %, a hydroxypropoxyl group content of, for example, from 3 to 12 wt. %, an acetyl group content of, for example, from 3 to 15 wt. %, and a succinoyl group content of, for example, from 2 to 20 wt. %.

Specific examples of the hydroxypropylmethyl cellulose acetate succinate (HPMCAS) include those having a methoxyl group content of from 20 to 24 wt. %, a hydroxypropoxyl group content of from 5 to 9 wt. %, an acetyl group content of from 5 to 9 wt. %, and a succinoyl group content of from 14 to 18 wt. %. Examples of such commercially available products include Shin-Etsu AQOAT (registered trademark) grade AS-L. Other specific examples of the hydroxypropylmethyl cellulose acetate succinate (HPMCAS) include those having a methoxyl group content of from 21 to 25 wt. %, a hydroxypropoxyl group content of from 5 to 9 wt. %, an acetyl group content of from 7 to 11 wt. %, and a succinoyl group content of from 10 to 14 wt. %. Examples of such commercially available products include Shin-Etsu AQOAT (registered trademark) grade AS-M. Still other specific examples of the hydroxypropylmethyl cellulose acetate succinate (HPMCAS) include those having a methoxyl group content of from 22 to 26 wt. %, a hydroxypropoxyl group content of from 6 to 10 wt. %, an acetyl group content of from 10 to 14 wt. %, and a succinoyl group content of from 4 to 8 wt. %. Examples of such commercially available products include Shin-Etsu AQOAT (registered trademark) grade AS-H.

The water swelling rate of the component (D2) is, for example, 60% or less, preferably 40% or less, and more preferably 30% or less. The method for measuring the water swelling rate of the component (D2) is the same as the method for measuring the water swelling rate of the component (C).

The component (D2) to be used may be one type of the above-described compounds alone, or a combination of a plurality of types thereof.

### 1-4-3. Content of Component (D)

The content of the component (D) is not particularly limited as long as the effects of the present invention are achieved. The content of the component (D) per 1 part by weight of the total amount of the components (A) and (B) is, for example, from 0.01 to 5 parts by weight. From the viewpoint of further improving the moldability of the orally disintegrating tablet, the content of the component (D) per 1 part by weight of the total amount of the components (A) and (B) is preferably from 0.05 to 4 parts by weight, and more preferably from 0.1 to 3.5 parts by weight as the content of the component (D) per 1 part by weight of the total amount of the components (A) and (B).

The content of the component (D11) per 1 part by weight of the total amount of the components (A) and (B) is, for example, from 0.1 to 5 parts by weight, and from the viewpoint of further improving the moldability of the orally disintegrating tablet, the content thereof is preferably from 0.6 to 4 parts by weight, more preferably from 0.8 to 3.3 parts by weight, even more preferably from 1 to 2.6 parts by weight, and still more preferably from 1.5 to 2.4 parts by weight.

The content of the component (D13) per 1 part by weight of the total amount of the components (A) and (B) is, for example, from 0.01 to 3 parts by weight or from 0.03 to 2.5 parts by weight, and from the viewpoint of further improving the moldability of the orally disintegrating tablet, the content thereof is preferably from 0.05 to 2 parts by weight, and more preferably from 0.1 to 1.5 parts by weight. When the component (C) is crospovidone, the content of the component (D13) per 1 part by weight of the total amount of the components (A) and (B) is more preferably from 0.18 to 0.7 parts by weight, even more preferably from 0.25 to 0.6 parts by weight, and still even more preferably from 0.3 to 0.42 parts by weight. and when the component (C) is croscarmellose, the content of the component (D13) is from 0.1 to 1.5 parts by weight, from 0.1 to 1 part by weight, or from 0.1 to 0.5 parts by weight, and more preferably from 0.17 to 0.4 parts by weight or from 0.18 to 0.3 parts by weight.

The content of the component (D2) per 1 part by weight of the total amount of the components (A) and (B) is, for example, from 0.1 to 5 parts by weight, and from the viewpoint of further improving the moldability of the orally disintegrating tablet, the content thereof is preferably from 0.3 to 3 parts by weight, more preferably from 0.5 to 2 parts by weight, and even more preferably from 0.7 to 1 part by weight.

The content of the component (D) per 1 part by weight of the component (C) is, for example, from 0.1 to 50 parts by weight. From the viewpoint of further improving the moldability of the orally disintegrating tablet, the content of the component (D) per 1 part by weight of the component (C) is preferably from 0.5 to 50 parts by weight, more preferably from 1 to 45 parts by weight, even more preferably from 2.5 to 40 parts by weight, and still more preferably 2.5 to 35 parts by weight.

The content of the component (D11) per 1 part by weight of the component (C) is, for example, from 1 to 50 parts by weight, and from the viewpoint of further improving the moldability of the orally disintegrating tablet, the content thereof is preferably from 6 to 40 parts by weight, more preferably from 8 to 32 parts by weight, even more preferably from 10 to 25 parts by weight, and still more preferably from 15 to 20 parts by weight.

The content of the component (D13) per 1 part by weight of the component (C) is, for example, from 0.1 to 30 parts by weight, and from the viewpoint of further improving the moldability of the orally disintegrating tablet, the content thereof is preferably from 0.5 to 20 parts by weight, more preferably from 1 to 15 parts by weight, even more preferably from 2.5 to 7 parts by weight or from 2.5 to 6 parts by weight, and still more preferably from 3 to 4.2 parts by weight or from 3.2 to 4.2 parts by weight.

The content of the component (D2) per 1 part by weight of the component (C) is, for example, from 1 to 50 parts by weight, and from the viewpoint of further improving the moldability of the orally disintegrating tablet, the content thereof is preferably from 3 to 30 parts by weight, more preferably from 5 to 20 parts by weight, and even more preferably from 7 to 10 parts by weight.

The specific content of the component (D) in the additive composition for an orally disintegrating tablet according to an embodiment of the present disclosure is, for example, from 1 to 70 wt. %. From the viewpoint of further improving the moldability of the orally disintegrating tablet, the content of the component (D) is preferably from 5 to 70 wt. %, more preferably from 5 to 60 wt. %, even more preferably from 7 to 60 wt. %, and still more preferably from 9 to 60 wt. %, from 9 to 50 wt. %, from 9 to 40 wt. %, from 9 to 30 wt. %, or from 9 to 25 wt. %.

### 1-5. Additional Component (X)

The additive composition for an orally disintegrating tablet according to an embodiment of the present disclosure may consist only of the above-mentioned components (A), (B), (C) and (D), or may further contain an additional component (hereinafter, also referred to as the “component (X)”) other than the active pharmaceutical ingredient blended in the orally disintegrating tablet as long as the effects of the present invention are not impaired.

Examples of the component (X) include a base, a binder, an excipient, a disintegrant, a fluidizing agent, a surfactant, a sweetener, an acidulant, a flavoring agent, a fragrance, a colorant, and a stabilizer, other than the components (A), (B), (C) and (D). When such an additional component is contained in the present disclosure, preferred examples of the additional component include carmellose, magnesium stearate, sodium stearyl fumarate, and light silicic anhydride. A single type of these additional components may be used alone, or a plurality of types may be used in combination.

When the component (C) is crospovidone, the additive composition preferably contains at least magnesium stearate as the component (X), and when the component (C) is croscarmellose sodium, the additive composition preferably contains at least sodium stearyl fumarate as the component (X).

When the component (X) is contained, the total amount of the components (A), (B), (C), and (D) in the additive composition for oral disintegration according to an embodiment of the present disclosure is, for example, 40 wt. % or more and less than 100 wt. %, preferably 50 wt. % or more and less than 100 wt. % or 60 wt. % or more and less than 100 wt. %, more preferably 70 wt. % or more and less than 100 wt. % or 80 wt. % or more and less than 100 wt. %, and even more preferably 90 wt. % or more and less than 100 wt. % or 95 wt. % or more and less than 100 wt. %.

### 1-6. Production Method

The additive composition according to an embodiment of the present disclosure can be produced by any method or means known to a person skilled in the art. For example, the additive composition according to an embodiment of the present disclosure can be produced by mixing various components constituting the additive composition (that is, the components (A) to (D) and the component (X) added as necessary) all at one time.

The additive composition according to an embodiment of the present disclosure can also be produced using any granulation method. The granulation method is not particularly limited, and examples thereof include a dry granulation method and a wet granulation method.

The dry granulation method includes mixing powders of the various components to be contained in the additive composition, forming the mixture into small clumps by a strong pressure, and crushing and granulating the small clumps. Specific examples of the dry granulation method include a crushing granulation method and a roll compaction method.

The wet granulation method includes forming a composite by dispersing, in the presence of a liquid, the various components to be contained in the additive composition, followed by drying. The liquid is not particularly limited as long as it is a liquid that is acceptable for pharmaceuticals or foods, and examples thereof include solvents such as water, ethanol, methanol, and acetone, and an aqueous solution in which less than 10 wt. % of the components of the additive composition are dissolved. Among these liquids, water or an aqueous solution is particularly preferred.

Specific examples of the wet granulation method include a spray drying method, a tumbling granulation method, a stirring granulation method, a fluidized bed granulation method, a freeze drying method, and a kneading granulation method.

When the additive composition according to an embodiment of the present disclosure is produced using a wet granulation method, the wet granulation method to be used may be a single-stage granulation method in which all of the various components to be contained in the additive composition are granulated all at once, or may be a multi-stage granulation method in which the various components are granulated in a plurality of stages. With regard to the multi-stage granulation method, as appropriate and according to details such as the type and amount of each component, a person skilled in the art could determine which component to select for use in each stage, from among the various components to be contained in the additive composition.

Furthermore, the conditions to be applied in each granulation process, such as spraying rate, air feed temperature, exhaust temperature, and air feed amount can be appropriately determined by a person skilled in the art according to details such as the type and amount of each component to be granulated.

In a preferred example of a case where the additive composition according to an embodiment of the present disclosure is produced using any granulation method, the additive composition can be produced by preparing a granulated product containing some of the constituent components of the additive composition (specifically, the components (A) to (D) and the component (X) that is added as necessary), and then mixing the prepared granulated product with the remaining constituent components of the additive composition. Examples of the some of the constituent components of the additive composition include the above-described components (A) to (C) and the component (X) that is added as necessary. Preferably, when the component (C) is crospovidone, examples of the some of the constituent components of the additive composition include the components (A) to (C) and the component (X), and when the component (C) is croscarmellose sodium, examples of the some of the constituent components of the additive composition include the components (A) to (C).

### 2. Orally Disintegrating Tablet Composition

The orally disintegrating tablet composition according to an embodiment of the present disclosure contains the above-mentioned “1. Additive Composition for Orally Disintegrating Tablet” and an active pharmaceutical ingredient (E) (also referred to as API, and hereinafter, may also be referred to as the “component (E)”).

The component (E) is not particularly limited as long as it is an active pharmaceutical ingredient that is blended into an orally disintegrating tablet and taken. Such an active pharmaceutical ingredient generally lowers compression moldability regardless of the type, and therefore is not particularly limited. In particular, known examples include active pharmaceutical ingredients having poor compression moldability such as acetaminophen and ascorbic acid. Furthermore, applications and types of the active pharmaceutical ingredient contained in the orally disintegrating tablet are not particularly limited and include, for example, drugs for the central nervous system, drugs for the peripheral nervous system, drugs for sensory organs, drugs for circulatory organs, drugs for respiratory organs, drugs for digestive organs, hormones, urogenital drugs, pharmaceuticals for other individual organs, vitamins, analeptics, drugs for blood and bodily fluids, other metabolic pharmaceuticals, drugs for cellular activation, tumor drugs, radioactive pharmaceuticals, drugs for allergies, other pharmaceuticals for tissue cell function, herbal medicines, Chinese medicines, other pharmaceuticals based on herbal and Chinese medicines, antibiotic formulations, chemotherapeutic drugs, biological formulations, drugs for parasites, pharmaceuticals for other pathogenic organisms, drugs for preparations, diagnostic drugs, drugs for public health, and pharmaceuticals for extracorporeal diagnosis. A single type of the active pharmaceutical ingredient may be used alone, or two or more types may be used in combination.

The form of the component (E) is also not particularly limited, and examples thereof include powders; granulated products obtained by granulation for the purpose of improving content uniformity and the like; and coated particles whose surface is wholly or partially coated to impart functions such as bitterness masking, gastric solubility, enteric solubility, and sustained-release properties.

The content of the component (E) in the orally disintegrating tablet composition according to an embodiment of the present disclosure is not particularly limited, and the content of the component (E) per 100 parts by weight of the additive composition for an orally disintegrating tablet is, for example, from 0.5 to 233 parts by weight, preferably from 1 to 200 parts by weight, more preferably from 5 to 150 parts by weight, and even more preferably from 11 to 150 parts by weight. The lower limit of the range of the content of the component (E) may be 20 parts by weight or more, 30 parts by weight or more, 40 parts by weight or more, 50 parts by weight or more, 60 parts by weight or more, or 65 parts by weight or more. The upper limit of the range of the content of the component (E) may be 100 parts by weight or less, 80 parts by weight or less, or 70 parts by weight or less.

In terms of the content of the component (E) per 100 parts by weight of the total amount of the components (A), (B), (C), and (D) in the additive composition for an orally disintegrating tablet, the content of component (E) in the orally disintegrating tablet composition according to an embodiment of the present disclosure may be, for example, from 0.5 to 233 parts by weight, preferably from 1 to 200 parts by weight, more preferably from 5 to 150 parts by weight, and even more preferably from 11 to 150 parts by weight. The lower limit of the range of the content of the component (E) may be 20 parts by weight or more, 30 parts by weight or more, 40 parts by weight or more, 50 parts by weight or more, 60 parts by weight or more, or 65 parts by weight or more. The upper limit of the range of the content of the component (E) may be 100 parts by weight or less, 80 parts by weight or less, or 70 parts by weight or less.

### 3. Orally Disintegrating Tablet

The orally disintegrating tablet according to an embodiment of the present disclosure is a tablet produced by tableting the above-described “2. Orally Disintegrating Tablet Composition”. The orally disintegrating tablet according to an embodiment of the present disclosure exhibits an excellent balance between high tablet hardness and short disintegration time.

The weight per tablet of the orally disintegrating tablet according to an embodiment of the present disclosure is, for example, from 50 to 500 mg, preferably from 100 to 460 mg, more preferably from 150 to 440 mg, and even more preferably from 180 to 420 mg, from 180 to 420 mm, from 220 to 420 mg, from 320 to 420 mg, or from 180 to 320 mg.

### 4. Method for Producing Orally Disintegrating Tablet

The method for producing an orally disintegrating tablet according to an embodiment of the present disclosure includes: preparing an orally disintegrating tablet composition by mixing (A) an excipient selected from the group consisting of a water-soluble saccharide and a sugar alcohol, (B) a crystalline cellulose, (C) a swellable water-insoluble polymer selected from the group consisting of crospovidone and croscarmellose sodium, (D) (D1) a (meth)acrylic polymer and/or (D2) a cellulosic polymer selected from the group consisting of ethyl cellulose, cellulose acetate, hydroxypropylmethyl cellulose acetate succinate, and hydroxypropylmethyl cellulose phthalate, and (E) an active pharmaceutical ingredient; and tableting the orally disintegrating tablet composition.

The method for producing an orally disintegrating tablet according to an embodiment of the present disclosure involves the use of the additive composition described in “1. Additive Composition for Orally Disintegrating Tablet” above, and therefore the moldability of the orally disintegrating tablet is improved. Accordingly, an orally disintegrating tablet having a high hardness can be molded by a relatively small tableting compression force, and thus an orally disintegrating tablet having an appropriate void rate and an excellent disintegrability can be produced.

Specifically, the tableting pressure used for tableting in the method for producing an orally disintegrating tablet according to an embodiment of the present disclosure is, for example, from 20 to 600 MPa, preferably from 40 to 400 MPa, and more preferably from 80 to 300 MPa.

Each aspect disclosed in the present specification can be combined with any other feature disclosed herein.

### EXAMPLES

Hereinafter, the present invention will be more specifically described by way of examples, but each of the configurations, the combinations thereof, and the like in each embodiment are merely examples, and various additions, omissions, substitutions, and other changes of configurations may be made as appropriate without departing from the spirit of the present invention. The present disclosure is not limited by the embodiments and is limited only by the claims.

### 1. Preparation of Additive Composition for Orally Disintegrating Tablet or Orally Disintegrating Tablet Composition

There was prepared an additive composition for an orally disintegrating tablet containing the components (A) to (D) and the component (X) shown in Table 1, or an orally disintegrating tablet composition containing the components (A) to (D), the component (X), and the component (E) shown in Table 1. The water swelling rate of the component (C) was calculated. Specifically, 75 mL of purified water was added to a beaker, and 5.0 g of the component (C) was added a little at a time under stirring with a stirrer. After completion of the addition of the component, the mixture was stirred for 3 minutes. The resultant suspension was transferred to a 100 mL measuring cylinder, and the volume was increased to 100 mL, after which the suspension was allowed to stand for 16 hours, and then the volume after swelling was read. The read volume was substituted into the following formula to calculate the swelling rate.

$$Swelling rate [ \% ] = ( volume [ mL ] after swelling - volume [ mL ] before swelling ) / \\ ( volume [ mL ] before swelling ) \times 100 \qquad [ Math . 2 ]$$

TABLE 1

| (A) | | Mannitol | D-mannitol |
|---|---|---|---|
| (B) | | Crystalline cellulose | Ceolus (available from Asahi Kasei Corp.) |
| (C) | | Crospovidone | Polyplasdone INF-10 (available from ASHLAND |
| | | | Inc.), swelling rate 80% |
| | | Croscarmellose | Ac-di-sol (available from FMC Corporation), swelling |
| | | sodium | rate 744% |
| (D) | (D1) | (D11) Methacrylic | Eudragit (registered trademark) L100-55, copolymer |
| | | acid copolymer | of ethyl acrylate and methacrylic acid at |
| | | | polymerization ratio of 1:1 |
| | | (D13) Aminoalkyl | Eudragit (registered trademark) EPO, copolymer of |
| | | methacrylate | butyl methacrylate, (2-dimethylaminoethyl) |
| | | copolymer | methacrylate, and methyl methacrylate at 1:2:1 |
| | (D2) | Ethyl cellulose | EC-N50 (available from The Dow Chemical |
| | | | Company) |
| | | Cellulose acetate | CA-398-10NF (available from Eastman Chemical |
| | | | Company) |

| (X) | Carmellose | Carmellose NS-300 (available from Nichirin Chemical |
|---|---|---|
| | | Industries, Ltd.) |
| | Magnesium stearate | Available from Taihei Chemical Industrial Co., Ltd. |
| | Sodium stearyl | PRUV (JRS Pharma GmbH & Co., KG) |
| | fumarate | |
| (E) | Acetaminophen | Available from Spera Nexus, Inc. |
| | Ascorbic acid | Available from Iwaki Seiyaku Co., Ltd. |

### 1-1. Comparative Examples 1 and 2 and Examples 1 to 7

### Production of Granulated Product 1

280 g of mannitol, 75 g of carmellose, 100 g of crystalline cellulose, and 40 g of crospovidone were added to a fluidized bed granulator (FL-LABO, Powrex Corp.) and sprayed with 300 g of purified water at a rate of 12 g/min, to produce a granulated product 1.

### Example 1

To 89.5 parts by weight of the produced granulated product 1 were added 10.0 parts by weight of the aminoalkyl methacrylate copolymer and 0.5 parts by weight of magnesium stearate (available from Taihei Chemical Industrial Co., Ltd.), and the mixture was tableted using a simple tableting machine (HANDTAB-100, available from Ichihashi Seiki Kyoto Japan) at a tableting pressure of 59.7 MPa, to produce an angled-corner flat tablet having a diameter of 8.0 mm and a weight of 200 mg.

### Example 2

To 79.5 parts by weight of the produced granulated product 1 were added 20.0 parts by weight of the aminoalkyl methacrylate copolymer and 0.5 parts by weight of magnesium stearate (available from Taihei Chemical Industrial Co., Ltd.), and the mixture was tableted using a simple tableting machine (HANDTAB-100, available from Ichihashi Seiki Kyoto Japan) at a tableting pressure of 39.8 MPa, to produce an angled-corner flat tablet having a diameter of 8.0 mm and a weight of 200 mg.

### Example 3

To 59.5 parts by weight of the produced granulated product 1 were added 40.0 parts by weight of the methacrylic acid copolymer and 0.5 parts by weight of magnesium stearate (available from Taihei Chemical Industrial Co., Ltd.), and the mixture was tableted using a simple tableting machine (HANDTAB-100, available from Ichihashi Seiki Kyoto Japan) at a tableting pressure of 79.6 MPa, to produce an angled-corner flat tablet having a diameter of 8.0 mm and a weight of 200 mg.

### Example 4

To 39.5 parts by weight of the produced granulated product 1 were added 60.0 parts by weight of the methacrylic acid copolymer and 0.5 parts by weight of magnesium stearate (available from Taihei Chemical Industrial Co., Ltd.), and the mixture was tableted using a simple tableting machine (HANDTAB-100, available from Ichihashi Seiki Kyoto Japan) at a tableting pressure of 59.7 MPa, to produce an angled-corner flat tablet having a diameter of 8.0 mm and a weight of 200 mg.

### Comparative Example 1

To 99.5 parts by weight of the produced granulated product 1 was added 0.5 parts by weight of magnesium stearate (Taihei Chemical Industrial Co., Ltd.), and the mixture was tableted using a simple tableting machine (HANDTAB-100, available from Ichihashi Seiki Kyoto Japan) at a tableting pressure of 119.4 MPa, to produce an angled-corner flat tablet having a diameter of 8.0 mm and a weight of 200 mg.

### Production of Granulated Product 2

220 g of mannitol, 160 g of crystalline cellulose, and 20 g of croscarmellose sodium were added to a fluidized bed granulator (FL-LABO, Powrex Corp.) and sprayed with 600 g of purified water at a rate of 12 g/min, to produce a granulated product 2.

### Example 5

To 89.0 parts by weight of the produced granulated product 2 were added 10.0 parts by weight of the aminoalkyl methacrylate copolymer and 1.0 part by weight of sodium stearyl fumarate (SSF) (PRUV, available from JRS Pharma GmbH & Co., KG), and the mixture was tableted using a simple tableting machine (HANDTAB-100, available from Ichihashi Seiki Kyoto Japan) at a tableting pressure of 79.6 MPa, to produce an angled-corner flat tablet having a diameter of 8.0 mm and a weight of 200 mg.

### Example 6

To 79.0 parts by weight of the produced granulated product 2 were added 20.0 parts by weight of the aminoalkyl methacrylate copolymer and 1.0 part by weight of sodium stearyl fumarate (SSF) (PRUV, available from JRS Pharma GmbH & Co., KG), and the mixture was tableted using a simple tableting machine (HANDTAB-100, available from Ichihashi Seiki Kyoto Japan) at a tableting pressure of 59.7 MPa, to produce an angled-corner flat tablet having a diameter of 8.0 mm and a weight of 200 mg.

### Example 7

To 39.0 parts by weight of the produced granulated product 2 were added 60.0 parts by weight of the methacrylic acid copolymer and 1.0 part by weight of sodium stearyl fumarate (SSF) (PRUV, available from JRS Pharma GmbH & Co., KG), and the mixture was tableted using a simple tableting machine (HANDTAB-100, available from Ichihashi Seiki Kyoto Japan) at a tableting pressure of 79.6 MPa, to produce an angled-corner flat tablet having a diameter of 8.0 mm and a weight of 200 mg.

### Comparative Example 2

To 99.0 parts by weight of the produced granulated product 2 was added 1.0 part by weight of sodium stearyl fumarate (SSF) (PRUV, available from JRS Pharma GmbH & Co., KG), and the mixture was tableted using a simple tableting machine (HANDTAB-100, available from Ichihashi Seiki Kyoto Japan) at a tableting pressure of 119.4 MPa, to produce an angled-corner flat tablet having a diameter of 8.0 mm and a weight of 200 mg.

### 1-2. Examples 8 to 15, 19, and 20

### 1-2-1. Cases Using Granulated Product 1 or 2 (Examples 8 to 10, 13 to 15, and 19)

Tablets were produced in the same manner as in “1-1. Comparative Examples 1 and 2 and Examples 1 to 7” described above except that the conditions including tablet composition and tableting condition were changed as shown in Table 2A, Table 2B, and Table 3A.

### 1-2-2. Cases Using Neither Granulated Product 1 Nor Granulated Product 2 (Examples 11, 12, and 20)

Tablets were produced in the same manner as in “1-1. Comparative Examples 1 and 2 and Examples 1 to 7” described above except that granulation was not performed, and the conditions including tablet composition and tableting condition were changed as shown in Table 2A and Table 3A.

### 1-3. Comparative Examples 3 to 5 and Examples 16 to 18, 21, and 22

Tablets containing the additive composition and the component (E) were produced. Specifically, tablets were produced in the same manner as in “1-1. Comparative Examples 1 and 2 and Examples 1 to 7” described above except that the conditions including tablet composition and tableting condition were changed as shown in Table 2C and Table 3B.

### 2. Measurement of Tablet Properties

The following properties were measured for the produced tablets. The results are shown in Tables 2A, 2B, and 2C and Tables 3A and 3B.
(1) Hardness: the hardness (N) was measured using a digital Kiya hardness tester (KHT-40N, Fujiwara Scientific Co., Ltd.), and the hardness was determined according to the formula described below. The hardness was measured six times, and the average of the measured values was used as the measurement result.
(2) Disintegration time in water: the disintegration time in water (seconds) (hereinafter, also referred to simply as the “disintegration time”) was measured using a disintegration tester (NT-4HF, Toyama Sangyo Co., Ltd.) in accordance with a method described in the Japanese Pharmacopoeia (without disk). The disintegration time was measured six times, and the average of the measured values was used as the measurement result.

### 3. Evaluation of Moldability

The hardness of the tablet was converted into tensile strength on the basis of the following formula, and a value obtained by dividing the tensile strength by the tableting pressure was used as the evaluation value of moldability.

$$Tensile strength ( MPa ) = ( 2 \times tablet hardness [ N ] ) / ( π \times tablet thickness [ mm ] \times tablet diameter [ mm ] ) \qquad [ Math . 3 ]$$

$$Moldability evaluation value = tensile strength / tableting pressure$$

Moldability can be evaluated as being improved when the moldability evaluation value is larger than that in the case where the component (D) is not contained. Moldability can be evaluated as being more excellent when the moldability evaluation value is larger. The results are shown in Tables 2A, 2B, and 2C and Tables 3A and 3B.

TABLE 2A

| | | | Comparative | Example | Example | Example | Example | Example | Example | Example |
|---|---|---|---|---|---|---|---|---|---|---|
| | | | Example 1 | 8 | 9 | 10 | 1 | 2 | 11 | 12 |
| Granulated | (A) | Mannitol | (56.3) | (55.7) | (55.1) | (54.6) | (50.6) | (45) | 45 | 45 |
| product 1 | (B) | Crystalline | (20.1) | (19.9) | (19.7) | (19.5) | (18.1) | (16.1) | 16.1 | 16.1 |
| | | cellulose | | | | | | | | |
| | (C) | Crospovidone | (8) | (8.0) | (7.9) | (7.8) | (7.2) | (6.4) | 6.4 | 6.4 |
| | (X) | Carmellose | (15.1) | (14.9) | (14.8) | (14.6) | (13.6) | (12) | 12 | 12 |
| | | | 99.5 | 98.5 | 97.5 | 96.5 | 89.5 | 79.5 | — | — |
| (D) | (D1) | (D11) | — | — | — | — | — | — | — | — |
| | | Methacrylic | | | | | | | | |
| | | acid copolymer | | | | | | | | |
| | | (D13) | — | 1 | 2 | 3 | 10 | 20 | 20 | 20 |
| | | Aminoalkyl | | | | | | | | |
| | | methacrylate | | | | | | | | |
| | | copolymer | | | | | | | | |
| | (D2) | Ethyl cellulose | — | — | — | — | — | — | — | — |
| | | Cellulose | — | — | — | — | — | — | — | — |
| | | acetate | | | | | | | | |
| | (X) | Magnesium | 0.5 | 0.5 | 0.5 | 0.5 | 0.5 | 0.5 | 0.5 | 0.5 |
| | | stearate | | | | | | | | |

| | | | | | | | | |
|---|---|---|---|---|---|---|---|---|
| Additive composition total | 100 | 100 | 100 | 100 | 100 | 100 | 100 | 100 |
| [1](C)/{(A) + (B) + (C)} 100 | 9.5 | 9.5 | 9.5 | 9.5 | 9.5 | 9.5 | 9.5 | 9.5 |
| parts by weight | | | | | | | | |
| [2] (D)/{(A) + (B)} | 0 | 0.013 | 0.027 | 0.040 | 0.15 | 0.33 | 0.33 | 0.33 |
| [3] (D)/(C) | 0 | 0.13 | 0.25 | 0.38 | 1.39 | 3.13 | 3.13 | 3.13 |
| Tableting pressure [MPa] | 119.4 | 119.4 | 119.4 | 99.5 | 59.7 | 39.8 | 40.7 | 25.5 |
| Tablet diameter [mm] | 8.0 | 8.0 | 8.0 | 8.0 | 8.0 | 8.0 | 10.0 | 10.0 |
| Tablet weight [mg] | 200 | 200 | 200 | 200 | 200 | 200 | 300 | 300 |
| Tablet hardness [N] | 55.0 | 57.5 | 64 | 58.5 | 79.1 | 83.5 | 87.8 | 45.1 |
| Disintegration time in water | 13 | 14 | 15 | 13 | 9 | 10 | 13 | 19 |
| [sec] | | | | | | | | |
| Moldability | 0.011 | 0.012 | 0.013 | 0.014 | 0.029 | 0.043 | 0.037 | 0.028 |

In the table, the unit of a numerical value indicating the blended amount of each component is wt. %.
Note
that the unit of a proportion in [1] to [3] is parts by weight.
In the table, a numerical value in parentheses indicates the content (wt. %) of the corresponding component contained in the granulated product 1.

TABLE 2B

| | | | Comparative | Example | Example | Example | Example | Example |
|---|---|---|---|---|---|---|---|---|
| | | | Example 1 | 3 | 4 | 13 | 14 | 15 |
| Granulated | (A) | Mannitol | (56.3) | (33.7) | (22.3) | (16.4) | (33.7) | (33.7) |
| product 1 | (B) | Crystalline | (20.1) | (12) | (8) | (5.8) | (12) | (12) |
| | | cellulose | | | | | | |
| | (C) | Crospovidone | (8) | (4.8) | (3.2) | (2.3) | (4.8) | (4.8) |
| | (X) | Carmellose | (15.1) | (9) | (6) | (4.4) | (9) | (9) |
| | | | 99.5 | 59.5 | 39.5 | 29 | 59.5 | 59.5 |
| (D) | (D1) | (D11) | — | 40 | 60 | 70 | — | — |
| | | Methacrylic acid | | | | | | |
| | | copolymer | | | | | | |
| | | (D13) | — | — | — | — | — | — |
| | | Aminoalkyl | | | | | | |
| | | methacrylate | | | | | | |
| | | copolymer | | | | | | |
| | (D2) | Ethyl cellulose | — | — | — | — | 40 | — |
| | | Cellulose acetate | — | — | — | — | — | 40 |

| | | | | | | | |
|---|---|---|---|---|---|---|---|
| (X) | Magnesium | 0.5 | 0.5 | 0.5 | 0.5 | 0.5 | 0.5 |
| | stearate | | | | | | |

| | | | | | | |
|---|---|---|---|---|---|---|
| Additive composition total | 100 | 100 | 100 | 100 | 100 | 100 |
| [1](C)/{(A) + (B) + (C)} 100 parts | 9.5 | 9.5 | 9.5 | 9.5 | 9.5 | 9.5 |
| by weight | | | | | | |
| [2] (D)/{(A) + (B)} | 0 | 0.88 | 1.98 | 3.15 | 0.88 | 0.88 |
| [3] (D)/(C) | 0 | 8.33 | 18.75 | 30.43 | 8.33 | 8.33 |
| Tableting pressure [MPa] | 119.4 | 79.6 | 59.7 | 59.7 | 79.6 | 79.6 |
| Tablet diameter [mm] | 8.0 | 8.0 | 8.0 | 8.0 | 8.0 | 8.0 |
| Tablet weight [mg] | 200 | 200 | 200 | 200 | 200 | 200 |
| Tablet hardness [N] | 55.0 | 68.0 | 67.6 | 52.2 | 79.1 | 72 |
| Disintegration time in water [sec] | 13 | 11 | 11 | 10 | 9 | 9 |
| Moldability | 0.011 | 0.018 | 0.022 | 0.017 | 0.029 | 0.020 |

In the table, the unit of a numerical value indicating the blended amount of each component is wt. %.
Note
that the unit of a proportion in [1] to [3] is parts by weight.
In the table, a numerical value in parentheses indicates the content (wt. %) of the corresponding component contained in the granulated product 1.

TABLE 2C

| | | | Comparative | Example | Example | Comparative | Example |
|---|---|---|---|---|---|---|---|
| | | | Example 3 | 16 | 17 | Example 4 | 18 |
| Granulated | (A) | Mannitol | (56.2) | (55.5) | (44.9) | (56.2) | (55.5) |
| product 1 | (B) | Crystalline | (20.1) | (19.9) | (16.0) | (20.1) | (19.9) |
| | | cellulose | | | | | |
| | (C) | Crospovidone | (8.0) | (8.0) | (6.3) | (8.0) | (8.0) |
| | (X) | Carmellose | (15.1) | (14.9) | (12.0) | (15.1) | (14.9) |
| | | | 99.4 | 98.3 | 79.2 | 99.4 | 98.3 |
| (D) | (D1) | (D11) | — | — | — | — | — |
| | | Methacrylic acid | | | | | |
| | | copolymer | | | | | |
| | | (D13) | — | 1.1 | 20 | — | 1.1 |
| | | Aminoalkyl | | | | | |
| | | methacrylate | | | | | |
| | | copolymer | | | | | |
| | (D2) | Ethyl cellulose | — | — | — | — | — |
| | | Cellulose acetate | — | — | — | — | — |

| (X) | Magnesium | 0.6 | 0.6 | 0.8 | 0.6 | 0.6 |
|---|---|---|---|---|---|---|
| | stearate | | | | | |
| Additive composition total | | 100 | 100 | 100 | 100 | 100 |
| (E) | Acetaminophen | 11.1 | 11.1 | 66.7 | — | — |
| | Ascorbic acid | — | — | — | 11.1 | 11.1 |

| | | | | | |
|---|---|---|---|---|---|
| [1] (C)/{(A) + (B) + (C)} 100 parts | 9.5 | 9.5 | 9.5 | 9.5 | 9.5 |
| by weight | | | | | |
| [2] (D)/{(A) + (B)} | 0 | 0.015 | 0.33 | 0 | 0.015 |
| [3] (D)/(C) | 0 | 0.14 | 3.17 | 0 | 0.14 |
| [4] (E)/{(A) + (B) + (C) + (D)} | — | 13.1 | 76.5 | — | 13.1 |
| 100 parts by weight | | | | | |
| Tableting pressure [MPa] | 203.4 | 132.6 | 44.2 | 203.4 | 132.6 |
| Tablet diameter [mm] | 12.0 | 12.0 | 12.0 | 12.0 | 12.0 |
| Tablet weight [mg] | 400 | 400 | 400 | 400 | 400 |
| Tablet hardness [N] | 33.7 | 61.4 | 59.0 | 76.3 | 64.8 |
| Disintegration time in water [sec] | 25 | 17 | 9 | 17 | 23 |
| Moldability | 0.003 | 0.008 | 0.020 | 0.007 | 0.010 |

In the table, the units of a numerical value indicating the blended amount of each component and a proportion in [1] to [4] are parts by weight.
In the table, a numerical value in parentheses indicates the content (parts by weight) of the corresponding component contained in the granulated product 1.

TABLE 3A

| | | | Comparative | Example | Example | Example | Example | Example |
|---|---|---|---|---|---|---|---|---|
| | | | Example 2 | 5 | 19 | 6 | 20 | 7 |
| Granulated | (A) | Mannitol | (54.4) | (49) | (45.6) | (43.4) | 43.4 | (21.4) |
| product 2 | (B) | Crystalline | (39.6) | (35.5) | (33.1) | (31.6) | 31.6 | (15.6) |
| | | cellulose | | | | | | |
| | (C) | Croscarmellose | (5) | (4.5) | (4.1) | (4) | 4 | (2) |
| | | sodium | | | | | | |
| | | | 99.0 | 89.0 | 82.8 | 79.0 | — | 39.0 |
| (D) | (D1) | (D11) | — | — | — | — | — | 60 |
| | | Methacrylic | | | | | | |
| | | acid copolymer | | | | | | |
| | | (D13) | — | 10 | 16.2 | 20 | 20 | — |
| | | Aminoalkyl | | | | | | |
| | | methacrylate | | | | | | |
| | | copolymer | | | | | | |

| | | | | | | | |
|---|---|---|---|---|---|---|---|
| (X) | Sodium stearyl | — | — | — | — | — | — |
| | fumarate | | | | | | |

| | | | | | | |
|---|---|---|---|---|---|---|
| Additive composition total | 100 | 100 | 100 | 100 | 100 | 100 |
| [1] (C)/{(A) + (B) + (C)} 100 | 5.0 | 5.0 | 5.0 | 5.0 | 5.0 | 5.0 |
| parts by weight | | | | | | |
| [2] (D)/{(A) + (B)} | 0 | 0.12 | 0.21 | 0.27 | 0.27 | 1.62 |
| [3] (D)/(C) | 0 | 2.22 | 3.95 | 5 | 5 | 30 |
| Tableting pressure [MPa] | 79.6 | 79.6 | 44.2 | 59.7 | 50.9 | 79.6 |
| Tablet diameter [mm] | 8.0 | 8.0 | 12.0 | 8.0 | 10.0 | 8.0 |
| Tablet weight [mg] | 200 | 200 | 400 | 200 | 300 | 200 |
| Tablet hardness [N] | 78.3 | 135.3 | 140.4 | 145.4 | 123.7 | 127.8 |
| Disintegration time in water | 14 | 14 | 14 | 18 | 15 | 16 |
| [sec] | | | | | | |
| Moldability | 0.024 | 0.041 | 0.077 | 0.058 | 0.045 | 0.034 |

In the table, the unit of a numerical value indicating the blended amount of each component is wt. %.
Note
that the unit of a proportion in [1] to [3] is parts by weight.
In the table, a numerical value in parentheses indicates the content (wt. %) of the corresponding component contained in the granulated product 2.

TABLE 3B

| | | | Comparative | Example | Example |
|---|---|---|---|---|---|
| | | | Example 5 | 21 | 22 |
| Granulated | (A) | Mannitol | (54.4) | (43.1) | (52.6) |
| product 2 | (B) | Crystalline cellulose | (39.6) | (31.3) | (38.2) |
| | (C) | Croscarmellose | (4.9) | (3.9) | (4.8) |
| | | sodium | | | |
| | | | 98.9 | 78.3 | 95.6 |
| (D) | (D1) | (D11) Methacrylic | — | — | — |
| | | acid copolymer | | | |
| | | (D13) Aminoalkyl | — | 20 | 3.3 |
| | | methacrylate | | | |
| | | copolymer | | | |

| (X) | Sodium stearyl | 1.1 | 1.7 | 1.1 |
|---|---|---|---|---|
| | fumarate | | | |
| Additive composition total | | 100 | 100 | 100 |
| (E) | Acetaminophen | — | 66.7 | — |
| | Ascorbic acid | 11.1 | — | 11.1 |

| | | | |
|---|---|---|---|
| [1] (C)/{(A) + (B) + (C)} | 5.0 | 5.0 | 5.0 |
| 100 parts by weight | | | |
| [2] (D)/{(A) + (B)} | 0 | 0.27 | 0.036 |
| [3] (D)/(C) | 0 | 5.1 | 0.69 |
| [4] (E)/{(A) + (B) + (C) + (D)} | — | 67.9 | 11.20 |
| 100 parts by weight | | | |
| Tableting pressure [MPa] | 88.4 | 44.2 | 44.2 |
| Tablet diameter [mm] | 12.0 | 12.0 | 12.0 |
| Tablet weight [mg] | 400 | 400 | 400 |
| Tablet hardness [N] | 86.3 | 72.7 | 59.1 |
| Disintegration time in water [sec] | 14 | 9 | 26 |
| Moldability | 0.018 | 0.031 | 0.022 |

In the table, the units of a numerical value indicating the blended amount of each component and a proportion in [1] to [4] are parts by weight.
In the table, a numerical value in parentheses indicates the content (parts by weight) of the corresponding component contained in the granulated product 2.

As is clear from the results shown in Table 2, in Examples 1 to 4, moldability and disintegrability are improved as compared with those in Comparative Example 1. As is clear from the results shown in Table 3, in Examples 5 to 7, moldability and disintegrability are improved as compared with those in Comparative Example 2.

1. An additive composition for an orally disintegrating tablet, the additive composition comprising:
(A) an excipient selected from the group consisting of a water-soluble saccharide and a sugar alcohol;
(B) a crystalline cellulose;
(C) a swellable water-insoluble polymer selected from the group consisting of crospovidone and croscarmellose sodium; and
(D) (D1) a (meth)acrylic polymer and/or (D2) a cellulosic polymer selected from the group consisting of ethyl cellulose, cellulose acetate, hydroxypropylmethyl cellulose acetate succinate, and hydroxypropylmethyl cellulose phthalate.

2. The additive composition for an orally disintegrating tablet according to claim 1, wherein the component (D1) is a (meth)acrylic copolymer containing [d11] an alkyl (meth)acrylate unit and [d12] a comonomer unit selected from the group consisting of (meth)acrylic acid, an alkyl (meth)acrylate, an aminoalkyl (meth)acrylate, and an ammonioalkyl (meth)acrylate.

3. The additive composition for an orally disintegrating tablet according to claim 1, wherein a content of the component (D) per 1 part by weight of a total amount of the component (A) and the component (B) is from 0.01 to 5 parts by weight.

4. The additive composition for an orally disintegrating tablet according to claim 1, wherein a content of the component (D) per 1 part by weight of the component (C) is from 0.1 to 50 parts by weight.

5. The additive composition for an orally disintegrating tablet according to claim 1, wherein a content of the component (D) is from 1 to 70 wt. %.

6. An orally disintegrating tablet composition comprising the additive composition for an orally disintegrating tablet described in claim 1 and (E) an active pharmaceutical ingredient.

7. The orally disintegrating tablet composition according to claim 6, wherein a content of the component (E) per 100 parts by weight of the additive composition is from 0.5 to 233 parts by weight.

8. An orally disintegrating tablet comprising the orally disintegrating tablet composition described in claim 6.

9. A method for producing an orally disintegrating tablet, the method comprising:
preparing an orally disintegrating tablet composition by mixing (A) an excipient selected from the group consisting of a water-soluble saccharide and a sugar alcohol, (B) a crystalline cellulose, (C) a swellable water-insoluble polymer selected from the group consisting of crospovidone and croscarmellose sodium, (D) (D1) a (meth)acrylic polymer and/or (D2) a cellulosic polymer selected from the group consisting of ethyl cellulose, cellulose acetate, hydroxypropylmethyl cellulose acetate succinate, and hydroxypropylmethyl cellulose phthalate, and (E) an active pharmaceutical ingredient; and
tableting the orally disintegrating tablet composition.

10. The method according to claim 9, wherein the tableting is carried out at a tableting pressure of from 20 to 600 MPa.
